(19) United States
(12) Patent Application Publication
(10) Pub. No.: US 2004/0044063 A1
(43) Pub. Date: Mar. 4, 2004

(54) SMA therapy and cell based assay for identifying therapies

(76) Inventors: Brent Stockwell, Boston, MA (US); David E. Root, Brookline, MA (US)

Correspondence Address: FISH & RICHARDSON PC, 225 FRANKLIN ST, BOSTON, MA, 02110, US

(21) Appl. No.: 10/453,143

(22) Filed: Jun. 2, 2003

Related U.S. Application Data
(60) Provisional application No. 60/385,433, filed on May 31, 2002.

(51) Int. Cl. A61K 31/404

(52) U.S. Cl. 514/418

(57) ABSTRACT

This invention relates to therapies for diseases involving splicing defects, such as spinal muscular atrophy (SMA), and methods to identify compounds for treating this disease. The invention specifically provides for therapies comprised of small molecule compounds identified by cell-based high-throughput screening assays. These assays utilize engineered splicing constructs that fuse pre-mRNA fragments to a reporter gene. The fragments contain exons and at least one intron of a gene mutated in such a way to cause disease. Additionally, the invention provides for methods to monitor the effects of drugs on splicing and gene expression in vivo, in transgenic animals.

### RELATED APPLICATIONS

This application claims the benefit of U.S. Provisional Application No. 60/385,433 filed on May 31, 2002, the contents of which are incorporated herein by reference.

### GOVERNMENT SUPPORT

The work described herein was carried out, at least in part, using funds from the U.S. government under grant number RO1 NS41665 awarded by the National Institutes of Health, National Institute of Neurological Disorders and Stroke (NINDS). The government may therefore have certain rights in the invention.

### BACKGROUND OF THE INVENTION

It is estimated that 15% of point mutations that result in a human genetic disease cause RNA splicing defects (Krawczak et al., Hum. Mol. Gen. 90:41-54, 1992). One example of such a disease is Spinal Muscular Atrophy (SMA), an autosomal recessive disorder caused from the degeneration of a-motor neurons of the spinal cord anterior horn, leading to progressive muscular atrophy, paralysis, respiratory failure and infant death (Wirth, Hum. Mutat. 15:228-237, 2000). The most severe form of SMA, called Werdnig-Hoffman syndrome (SMA type I) and the two milder forms (types II and III) are caused by deletions or mutations of the survival motor neurons 1 (SMN1) gene (Wirth, Hum. Mutat. 15:228-237, 2000; Lefebvre et al., Cell 80:155-165, 1995).

Humans have two copies of the SMN gene (SMN1 and SMN2), which are located in a 500-kilobase (kb) inverted repeat on chromosome 5q13 (Lefebvre et al., Cell 80:155-165, 1995). The two SMN genes differ by a translationally silent C to T mutation at nucleotide position 6 in exon 7 (codon 280) of SMN2, which alters the splicing pattern of this transcript (Monani et al. Hum. Mol. Genet. 8:1177-1183, 1999; Lorson et al., Proc. Natl. Acad. Sci. USA 96:6307-6311, 1999). The telomeric gene SMN1 primarily generates mRNA transcripts that are spliced to encode full-length SMN protein, while SMN2 transcripts are alternatively spliced such that exons 5 and/or 7 are removed. The full-length proteins produced by the two genes are identical.

In SMA type I patients, the SMN1 gene is deleted entirely and SMN2 is spliced such that only ˜30% of mRNAs are full-length, and 70% of spliced transcripts exclude exon 7. The SMN2 gene product is sufficient to maintain fetal development, but infected individuals manifest the symptoms of SMA early in life and typically die as infants.

### SUMMARY OF THE INVENTION

The invention provides compositions and methods for identifying and evaluating a drug for treating a disease characterized by one or more splicing defects. The methods include screening assays to identify a drug or drugs for treating the genetic disease Spinal Muscular Atrophy (SMA).

Accordingly, one aspect of the invention relates to methods of treating an individual, such as a human, having or at risk for having SMA by administering a compound having the formula:

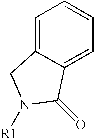

where R1 can be selected from the group consisting of:

C₁-C₂₅ alkyl, arylalkyl, substituted alkyl or substituted arylalkyl;

C₃-C₂₅ cycloalkyl, substituted cycloalkyl; arylcycloalkyl, or substituted arylcycloalkyl;

C₁-C₂₅ alkenyl, arylalkenyl, substituted alkenyl or substituted arylalkenyl;

C₃-C₂₅ cycloalkenyl, arylcycloakenyl, substituted cycloalkenyl, or substituted arylcycloalkenyl;

C₁-C₂₅ alkynyl, arylalkynyl, substituted alkynyl, or substituted arylalkynyl;

—COOH;

—COOR7, where R7 is independently selected from the same moieties as R1; and

—CONH₂

The residue R1 can have the following formula:

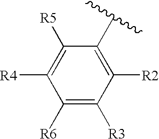

where each of R2, R3, R4, R5, and R6 is independently selected from hydrogen;

C₁-C₂₅ alkyl, arylalkyl, substituted alkyl or substituted arylalkyl;

C₃-C₂₅ cycloalkyl, substituted cycloalkyl; arylcycloalkyl, or substituted arylcycloalkyl;

C₁-C₂₅ alkenyl, arylalkenyl, substituted alkenyl or substituted arylalkenyl;

C₃-C₂₅ cycloalkenyl, arylcycloakenyl, substituted cycloalkenyl, or substituted arylcycloalkenyl;

C₁-C₂₅ alkynyl, arylalkynyl, substituted alkynyl, or substituted arylalkynyl;

—COOH;

—COOR7, where R7 is independently selected from the same moieties as R1; and

—CONH₂.

R6 can be a C1-C5 alcohol, a C1-C5 carboxylic acid, a C1-C5 ester, a C1-C5 aldehyde, or a C1-C5 ketone, such as

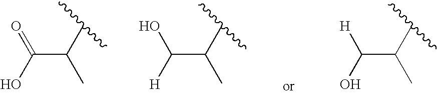

The compound can be, for example, indoprofen.

As used herein, the term “alkyl” refers to a hydrocarbon chain that may be a straight chain or branched chain, containing the indicated number of carbon atoms. For example, C₁-C₂₅ alkyl indicates that the group may have from 1 to 25 (inclusive) carbon atoms in it. The term “arylalkyl” refers to an alkyl moiety in which an alkyl hydrogen atom is replaced by an aryl group. Arylalkyl includes groups in which more than one hydrogen atom has been replaced by an aryl group. The point of attachment of the arylalkyl radical can be either through the aryl or alkyl portion of the arylalkyl group.

In one embodiment, the individual has or is at risk for having a deletion of or a mutation in the SMN1 gene. Administration of a compound described herein can modify the splicing pattern of SMN2 RNA, which can result in an increase in functional SMN2 protein levels.

One aspect of the invention relates to a method of treating a subject, such as a human, having or at risk for having spinal muscular atrophy (SMA) by administering a drug selected from the group consisting of indoprofen (Sigma-Aldrich); pyrithione zinc (Sigma-Aldrich); patulin (Sigma-Aldrich); camptothecin (Sigma-Aldrich); cefoxitin sodium (Sigma-Aldrich); amantadine (Sigma-Aldrich).

In either of these aspects, the subject typically has a deletion of, or a mutation in, the SMN1 gene. Without intending to be bound to any particular molecular mechanism, administration of such a drug can result in an increase in functional SMN2 protein levels, particularly in levels of SMN2 that include exon 7. The increase in SMN2 protein levels can be found in the cytoplasm and the nucleus, particularly in gems and Cajal bodies. Administration of a drug described herein can also result in increased levels of SMN2 oligomers. A drug described herein can treat a disease characterized by a splicing defect by preventing production of abnormal protein, by, for example, modifying the splicing pattern (e.g., correcting the splicing defect) or enhancing the stability of a transcript, selectively or non-selectively. Different drugs may operate by different mechanisms or by combinations of mechanisms. A candidate drug of the invention can be evaluated for an effect on splicing or transcript stability, or any other such mechanism that will result in the prevention or inhibition of abnormal protein production. For example, functional SMN protein can oligomerize or otherwise interact with a variety of other proteins including as ZPR1, SIP1 (Gemin-2), Gemin 3, Gemin 4, profilin II, Bcl-2 or the transactivator FUSE binding protein. Candidate drugs can be tested and/or evaluated for an effect of the interaction between SMN and one or more of these protein partners.

One aspect of the invention relates to methods for identifying a drug for treating a disease characterized by altered splicing of a pre-mRNA transcript (e.g., an SMN1 or SMN2 pre-mRNA). In one embodiment, the method includes providing a cell line that is transformed (e.g., stably transformed) with a transcribable cassette expressing at least a first and second exon and the intervening intron of a gene, such as an SMN1 or SMN2 gene. The second exon (or the 3′-most exon, if more than two exons are incorporated into the construct) can be fused to DNA encoding a detectable protein (i.e., a reporter protein) such as a luminescent or fluorescent protein. According to one embodiment, the cells of the cell line are exposed to a drug (e.g., a drug described herein), the cells are lysed by exposure to a lysing agent, the detectable protein is exposed to a substrate (if necessary to detect the reporter protein), and the cells are examined for the presence of the protein, as an indication that the transcript expressed from the transcribable cassette was appropriately spliced. An “appropriately spliced” transcript includes exon 2 fused to exon 1 so that the open reading frame is intact and allows expression of the reporter protein. The reporter protein can be, for example, luciferase, GFP, or YFP.

In one embodiment the reporter protein is luciferase and the substrate is D-luciferin. In a another embodiment, the luciferin is at a concentration less than 20 μM, and the pH of the lysing mixture is between about 7.5 and 8.2; in one embodiment the pH of the lysing mixture is 7.9. In other embodiments, the lysing agent comprises coenzyme A and dithiothreitol (DTT) at a concentration from about 8 mM to about 16 mM. For example, the concentration of DTT can be about 10 mM. The number of cells in an assay of the invention can be about 10,000 to about 20,000 per well of a 384-well plate, or from about 40,000 to about 80,000 per well of a 96-well plate. In one embodiment, before performing the luciferase-based assay, the cells can be protected from incident light for three hours or more. The embryonic mouse spinal cord cell line, NSC43, or the human cervical carcinoma cell line, C33A, are examples of cell lines that can be utilized for the assays described herein.

In one embodiment, the transcribable cassette includes all or fragments of exon 6, 7 and 8 and all or fragments of the intervening introns of the SMN1 or SMN2 genes.

In one embodiment of the invention, the transcribable cassette can express exons and intervening introns of any of the following genes: NF1, ATM, fibrillin (FBN1), dystrophin (DMD), BRCA1, TP53, MAPT, CD44, or CDKN2A.

In one embodiment the candidate drug is a small molecule, a peptide or an aptamer. A “small molecule” is a chemical compound that can affect the phenotype of a cell or organism by, for example, modulating the activity of a specific protein or nucleic acid within a cell. A “peptide” refers to a sequence of amino acids, generally shorter than a complete protein. An “aptamer” is a nucleic acid molecule having a tertiary structure that permits it to specifically bind to a protein ligand (see, e.g., Osborne, et al., Curr. Opin. Chem Biol. 1: 5-9, 1997; and Patel, Curr Opin Chem Biol 1:32-46, 1997). Since a nucleic acid molecule can in many cases be more conveniently introduced into target cells than a therapeutic protein molecule, aptamers offer a method by which splicing activity may be modulated without the introduction of chemical compounds or other small molecules which may have pluripotent effects. Generally, any of these types of therapeutic compositions can affect a cell, such as by altering a splicing pattern, such as by directly interacting with a protein or by interacting with a factor that acts upstream or downstream in a biochemical cascade that results in protein expression or activity, or that regulates splicing directly.

In one embodiment of the invention, a drug identified as a candidate for treating a disease is administered to a nonhuman transgenic animal, such as a mouse, rat, non-human primate, sheep, dog, cow, goat, or chicken, that is expressing an aberrantly spliced transcript. Following administration of the drug, one or more of the spliced transcripts can be detected, and the effect of the drug on the splicing pattern of the transcript can be a further indication of whether the drug would be a candidate to treat a disease.

This aspect of the invention is particularly adapted for high-throughput screening, in which the cell is exposed to a mixture including both a lysing agent and an enzyme substrate. Where the screen is directed to therapeutics for SMA, the cassette can contain a fragment of the SMN2 gene. Other diseases are described below (see Table 1). The cassette may include other exons from the gene, such as exon 8 of SMN2 (see below).

One aspect of the invention provides for a pharmaceutical composition that includes a drug identified by a method described herein, or a pharmaceutially acceptable salt or derivative thereof, in association with a pharmaceutically acceptable diluent or carrier.

One aspect of the invention provides a method of in vivo analysis of a drug for treating a disease characterized by an altered splicing pattern of a human gene as above. The method can include providing a non-human transgenic animal, such as a mouse, that includes a transcribable cassette with at least a first and second exon and an intervening intron, the second exon (the 3′-most exon) being fused to DNA encoding a reporter protein, such as a fluorescent or luminescent protein. The method can include administering a drug to the animal and detecting the reporter protein as an indication of the fidelity of splicing of exon 1 to exon 2. For example, detection of a reporter protein, such as a fluorescent protein (e.g., GFP or YFP) can be detected in mouse tissues, such as spinal cord or brain, by quantitative microscopy. Alternatively, a reporter protein can be detected by in situ analysis.

As used herein, a “transgenic animal” is a non-human animal, such as a mammal. The transgenic animal can be a rodent, such as a rat or mouse, in which one or more of the cells of the animal include a transgene. Other examples of transgenic animals include non-human primates, sheep, dogs, cows, goats, chickens, amphibians, and the like.

In one aspect of the invention, a transgenic animal, e.g., a transgenic mouse, is provided for assaying the effect of a drug on the splicing of an SMN1 or SMN2 cassette. For example, a transgenic mouse can carry a homozygous deletion of the SMN1 gene and the transcribable cassette can include exon 6 of SMN1 or SMN2 as the first exon, exon 7 as a second exon, and exon 8 as a third exon. A reporter gene, such as a gene encoding a fluorescent protein (e.g., YFP or GFP), can be fused to the third exon (exon 8). Expression of the reporter protein can be measured to monitor the fidelity of the splicing pattern, and consequently the effect of a drug on the splicing pattern. In one embodiment, expression of a fluorescent reporter protein can be assayed by quantitative fluorescence microscopy. In an alternative embodiment, a reporter protein can be a luminescent protein, such as luciferase, which can be assayed using a cooled charge coupled device or photomultiplier tube.

A fifth aspect of the invention relates to a method of in vivo analysis of a drug that alters the splicing pattern of a gene as above. In a preferred embodiment, the method includes providing a transgenic animal, such as a mouse or a rat, transformed with a transcribable cassette that includes at least a first and second exon and an intervening intron of a gene. The transgenic animal can be exposed to a drug, such as by feeding or injection (e.g., tail-vein injection or injection into the spinal cord) and the effect of the drug on splicing of the cassette can be assayed by, for example, RT-PCR, Northern blot analysis, or mRNA stability assay. The effect of the drug on the splicing pattern of the cassette can be an indication of whether the drug is a good candidate for treating the disease. For example, if the drug can alter the splicing pattern of an SMN2 cassette such that more full-length SMN2 mRNA is detected, then the drug can be a good candidate for treating SMA.

In one embodiment, an effect of the drug on protein production, such as reporter protein production or endogenous protein production, can be assayed. Protein levels can be assayed, for example, by Western blot, immunofluorescence, ELISA, or cytoblot. RNA processing can be monitored by standard molecular biology techniques, such as RT-PCR, Northern analysis, or an mRNA stability assay.

In another aspect, the invention provides methods to develop therapeutics for other diseases caused by mutations in a variety of genes (see the diseases listed in Table 1). The cassettes used in the assays described herein can be designed according to the sequences provided in the references listed in Table 1.

TABLE 1
Diseases caused by mutations that result in aberrant splicing

| Disease | Gene | Abberation | Reference |
|---|---|---|---|
| Neurofibromatosis | NF1 | germline mutation resulting in | Ars et al., Hum. Mol. Genet. |
| Type1 | | aberrant splicing (exon skipping or | 9:237-247, 2000 |
| | | activation of cryptic splice sites) | |
| ataxia telangiectasia | ATM | germline mutation resulting in | Teraoka et al., Am. J. Hum. |
| | | aberrant splicing (exon skipping or | Genet. 64:1617-1631, 1999. |
| | | activation of cryptic splice sites); eg: | |
| | | aberrant inclusion of a cryptic exon | |
| | | of 65 bp in one affected individual | |
| | | with a deletion of four nucleotides | |
| | | (GTAA) in intron 20. The deletion is | |
| | | located 12 bp downstream and 53 bp | |
| | | upstream from the 5′ and 3′ ends of | |
| | | the cryptic exon, respectively | |
| Marfan syndrome | Fibrillirin | nonsense mutation in exon 51 causes | Dietz et al., Science 259:680- |
| | (FBN1) | exon skipping | 683, 1993 |
| Duchenne muscular | Dystrophin | Mutations eliminate one or more | Ahn and Kunkel, Nature |
| dystrophy (DMD) | (DMD) | exon; eg. Nonsense E1211X in exon | Genet. 3:283-291, 1993 |
| | | 27 interrupts enhancer element | |
| | | which causes exon skipping | |
| Becker Muscular | Dystrophin | Mutations generally correlate with | Ahn and Kunkel, Nature |
| dystrophy (BMD) | (DMD) | maintenance of reading frame, or an | Genet. 3:283-291, 1993 |
| | | exon is skipped but reading frame is | |
| | | maintained | |
| Breast cancer | BRCA1 | Eg. E1694X nonsense mutation in | Mazoyer et al., Am. J. Hum. |
| | | exon 18 interrupts enhancer element | Genet. 62:713-715, 1998 |
| | | which causes exon skipping | |
| Cancer | TP53 | Tumor-associated silent point | Hernandez-Boussard et al., |
| | (encodes p53) | mutations are almost as numerous as | Hum. Mutat. 14:1-8, 1999 |
| | | nonsense mutations and often affect | |
| | | the same nucleotide; therefore effect | |
| | | is probably due to consequence on | |
| | | splicing pattern | |
| SMA | SMN1, | Homologous gene deletion; SMN2 | See references herein |
| | SMN2 | has silent point mutation in exon 7 | |
| FTDP17 | MAPT | (autosomal-dominant disorder | Lee et al., Annu. Rev. |
| (frontotemporal | (microtubule- | related to Alzheimer disease) | Neurosci. 24: 1121-1159, |
| dementia and | assoc. protein | mutations cluster around exon 10 | 2001. |
| parkinsonism | tau) | which encodes one of microtubule | |
| associated with | | binding motifs; alternative splicing | |
| chromosome 17) | | of exon 10 to produce specific | |
| | | isoforms is deregulated (end up with | |
| | | multiple effects on splicing pattern) | |
| Cancer | CD44 | Up to 4 different isoforms detected | See Noad et al. Adv Cancer |
| | | in primary tumors, only 2 known to | Res 71:241-319, 1997 and |
| | | exist in nontransformed tissue | references therein |
| Melanoma | CDKN2A | Eg: most common mutation | Harland et al., Human Mol. |
| | | identified in English families to | Genet. 10:2679-86, 2001 |
| | | date: A/G Pt mutation in middle of | |
| | | intron 2 creates a false GT splice | |
| | | donor site 105 bases 5′ of exon 3 and | |
| | | has been demonstrated to result in | |
| | | aberrant splicing of the mRNA. | |

One aspect of the invention provides a kit for the in vivo analysis of a compound to modify expression of a target gene. The kit can include, a reagent containing a lysing agent and a substrate for a luminescent protein. For example, the substrate can be D-luciferin to detect luciferase protein. In certain embodiments, the luciferin is present at a concentration of less than 20 μM, the pH of the reagent is between about 7.5 and 8.0 (e.g., pH 7.9), the reagent contains coenzyme A, and the reagent contains DTT at a concentration between about 4 mM and 15 mM (e.g., 10 mM). The kit can also incude an instruction manual.

One aspect of the invention provides a method for performing a business of screening for drugs that modify the splicing pattern of a gene. The method can include, for example, (a) accepting orders for performing screen for drugs that modify the splicing pattern of a gene designated by a user; (b) providing an in vitro screening system that includes a transcribable cassette having at least a first and second exon and an intervening intron, and where the second exon (or the 3′-most exon when more than 2 exons are required) is fused to a reporter gene, and a detection system; (c) recording the effect of each candidate drug in a library on the expression of the reporter gene in a print or computer-readable medium; and (d) providing the print or computer-readable medium to the user. In one embodiment, the library is provided by the user.

One aspect of the invention provides a method of identifying a drug for treating SMA. The method can include providing about 11,000 cells at about 200,000 cells/mL, wherein the cells are transformed (e.g., stably transformed) with a transcribable cassette that includes exons 6, 7, and a fragment of exon 8 of the SMN2 gene. Exon 8 can be fused in-frame to a luciferase reporter gene, and a single G nucleotide can be inserted at the 48^{th} position of exon 7. The cells can be protected from incident light for at least three hours prior to exposure to a candidate drug. Following exposure to the drug, the cells can be lysed by adding a mixture that includes a lysing agent, adjusted to a pH of about 7.85, D-luciferin, 10 mM dithiothreitol, Coenzyme A, and Triton X-100. Luminescence can then be measured. Detection of a change in luminescence following exposure to the drug can be an indication that the drug is a positive candidate for the treatment of SMA.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, useful methods and materials are described below. The materials, methods, and exemplification are illustrative only and not intended to be limiting. Other features and advantages of the invention will be apparent from the accompanying drawing and description, and from the claims.

### BRIEF DESCRIPTION OF THE DRAWINGS

FIG. 1 shows the structure of Indoprofen [4-(1,3-dihydro-1-oxo-isoindol-2-yl)-benzeneacetic acid].

FIG. 2 is a graph showing the effect of indoprofen on luminescence expressed from the SMN1-luciferase (squares) and SMN2-luciferase (diamonds) splicing constructs. The constructs are stably transformed into a C33A cell line. Also shown are the values for treated cell luminescence/control cell luminescence; treated cells were exposed to indoprofen and control cells were not treated with indoprofen. These values are representative of the increase in luminescence in the SMN2-luciferase transformed cells minus the increase in luminescence for the SMN1-luciferase transformed cells.

FIG. 3 is a graph showing the effect of pyrithione zinc on luminescence expressed from the SMN1-luciferase (squares) and SMN2-luciferase (diamonds) splicing constructs. The constructs are stably transformed into a C33A cell line. Also shown are the values for treated cell luminescence/control cell luminescence; treated cells were exposed to pyrithione zinc and control cells were not treated with pyrithione zinc. These values are representative of the increase in luminescence in the SMN2-luciferase transformed cells minus the increase in luminescence for the SMN1-luciferase transformed cells.

FIG. 4 is a graph showing the effect of patulin on luminescence expressed from the SMN1-luciferase (squares) and SMN2-luciferase (diamonds) splicing constructs. The constructs are stably transformed into a C33A cell line. Also shown are the values for treated cell luminescence/control cell luminescence; treated cells were exposed to patulin and control cells were not treated with patulin. These values are representative of the increase in luminescence in the SMN2-luciferase transformed cells minus the increase in luminescence for the SMN1-luciferase transformed cells.

### DETAILED DESCRIPTION

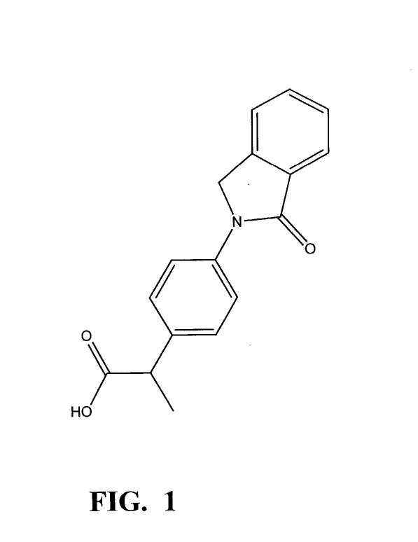

The present invention provides for methods and compositions for treating genetic diseases characterized by altered splicing patterns. In particular, a method is presented for treating SMA using the small molecule indoprofen (FIG. 1) and structural analogs of this compound. Indoprofen can be obtained commercially from Sigma-Aldrich. The invention also relates to methods of identifying drugs, including small molecules, compounds, aptamers, and the like, useful for treating a disease characterized by an altered splicing pattern.

Splicing Assays

The splicing assays of the invention are suitable for use in cells, such as a human or mouse cell line, or in transgenic animals, including mice, sheep, dogs, cows, goats, chickens, amphibians, and the like. A splicing assay of the invention requires a transcribable cassette that contains minimal sequences from the endogenous gene necessary to monitor splicing of specific exons of interest. Depending on the disease and gene of interest (see Table 1, for example), the transcribable cassette of the invention can included minimally, two exons and an intervening intron. Alternatively, the transcribably cassette can contain 3, 4, or 5 exons, or more, with intervening introns. The exons and intron can be cloned into the cassette in their entirety, or fragments containing sequences necessary for splicing can be cloned into the splicing cassette. For example, an internal fragment of the intron can be deleted, leaving the intron/exon splice junction and the branch point sequence in the intron intact. A sufficient amount of sequence flanking the splice junctions should be left intact to provide necessary regulatory elements and “mutated” sequence that may lead to abberrant splicing and thus cause disease, and most particularly, the disease of interest. Aberrant splicing can be manifested by splicing to cryptic splice sites in an exon or in the intron. This event can have a variety of effects on protein production, depending on the location of the exons on the gene. For example, aberrant splicing of an exon in the coding region of a pre-mRNA transcript can create a premature stop codon, which might lead to expression of a truncated protein. The truncated protein could be inactive, or it could have a different and/or antagonist function to the protein produced from the correctly spliced transcript. Aberrant splicing of an exon in te coding region can alternatively cause a frameshift mutation, which would alter the aino acid content of the C-terminal part of the protein. This can alter the structure and/or function of the protein. Aberrant splicing of a codon in either the upstream or downstream untranslated regions of the gene can lead to misregulation of expression, such as no expression (translation) at all; decreased or increased stability of the mRNA transcript, which can alter protein levels in the cell; or mislocalization of the mRNA transcript in the cell, which can lead to mislocalization of the protein in the cell.

To identify a drug (e.g., a compound or molecule) capable of modifying the splicing pattern of a gene, a construct as described above can be fused to a reporter gene, such as a gene encoding a fluorescent or luminescent protein, in order to monitor translation of the spliced construct in vivo. The reporter gene is fused in frame to the 3′-most exon. For example, if the construct contains two exons, the reporter gene is fused to the second exon; if the construct contains three exons, the reporter gene is fused to the third exon; etc. The endogenous exon to which the reporter gene is fused can be an exon fragment as long as the reporter gene is fused to the 3′ end of the exon. A measure in the change of reporter gene activity is a measure of the effect of a drug on the splicing pattern of the construct.

One exemplary splicing assay of the invention utilizes a splicing construct containing exons 6, 7, and 8 of the SMN2 gene, which has a translationally silent C to T mutation at nucleotide position 6 in exon 7. Exon 8 is truncated at its 3′ end by a fusion to a reporter gene, such as a gene encoding luciferase. An endogenous SMN2 gene is aberrantly spliced such that 30% of SMN2 mRNAs are full-length, and 70% of SMN2 mRNAs exclude exon 7. In an assay of the invention, a construct containing exons 6, 7, and 8 as described above will be translated to produce luciferase if exon 7 is spliced into the mRNA transcript. If exon 7 is spliced out of the mRNA, the reading frame is shifted, and no luciferase protein is produced. In the particular case of the SMN2 gene, exon 7 of the endogenous gene contains a stop codon, and exon 8 is a non-coding exon. In order to allow the reporter construct to function effectively as a reporter for splicing activity, one nucleotide can be inserted into exon 7 to eliminate the stop codon. Similar inert modifications can be applied depending on the particular gene to be examined. Care should be taken to avoid modifying nucleotides that fall into splice sites or splicing regulatory elements.

SMN Protein Activity

Full-length SMN protein is present in the cytoplasm and in small subnuclear bodies, including gems and Cajal bodies (also called coiled bodies) in most normal cells (Liu and Dreyfuss, EMBO J 15:3555-3556, 1996; Matera and Frey, Am. J. Hum. Gen. 63:317-321, 1998; Carvalho et al., J. Cell Biol. 147:715-728, 1999). Notably, the SMN-containing subnuclear bodies are not detected in some normal cell types including cardiac muscle, smooth muscle and spleen cells (Young et al., Exp. Cell Res. 256:365-374, 2000). Full-length SMN has also been reported to interact with transcription factors (Strasswimmer et al., Hum. Mol. Genet. 8: 1219-1226, 1999; Williams et al., FEBS Lett., 470:207-210, 1999), and the gem-associated profilins (Giesemann et al., J. Biol. Chem. 274:37908-37914, 1999). In addition, SMN binds to the zinc-finger protein ZPR1, the function of which is unknown, but cytoplasmic ZPR1 translocates with SMN1 to the subnuclear bodies upon treatment of mammalian cells with mitogens (Gelcheva-Gargova et al., Science 272:1797-1802, 1997). Truncated proteins translated from SMN2Δexon7 are found only in the cytoplasm (Frugier et al., Hum. Mol. Genet. 9:849-858, 2000). Further, SMN2Δexon7 does not interact with ZPR1, and in the absence of full-length SMN, ZPR fails to translocate to the nucleus of mammalian cells following treatment with mitogens (Gangwani et al., Nat. Cell Biol. 3:376-383, 2001). Other protein interactions interrupted by SMN2Δexon7 include SMN oligomerization (Lorson et al., Nature Genet. 19: 63-66, 1998) and the interaction of SMN with sm proteins within snRNPs (Pellizzoni et al., Proc. Natl. Acad. Sci. USA 96: 11167-11172, 1999). Interactions with the antiapoptotic protein Bcl-2 are disrupted, and in addition, the SMN2Δexon7 allele has a dominant negative effect on full-length SMN2 (Iwahashi et al., J. Cell. Biol. 148:1177-1186, 2000). The effect of a drug on SMN2 splicing can be assayed by the criteria discussed herein, including assays to test for the modulation of SMN2 protein interactions such as with transcription factors (e.g., the E2 protein of papilloma virus); profilin; ZPR1; sm proteins; Bcl-2; modulation of SMN2 oligomerization; and localization to the nucleus and/or nuclear bodies. The assays are alternatives to, or can be complementary to assays to test directly for an effect on splicing, including RT-PCR, Northern blot analysis, and in situ analysis of the SMN2 transcript. The assays are also alternatives to, or can be complementary to, the cell-based and transgenic animal-based experiments described herein, which utilize an SMN2 splicing construct fused to a reporter gene, for in vivo analyses.

Screening Methods

The invention provides for methods of identifying drugs that alter the splicing pattern of a gene (i.e., a pre-mRNA transcript) in a subject, such as a human or animal (e.g., a dog, cat, bird, cow, pig or other domestic animal), thereby treating a disease or condition. The candidate drugs can be chemical compounds, peptides, nucleic acid aptamers and the like.

Using the methods described herein, indoprofen was identified in a cell-based screen of a chemical library containing 2500-3000 compounds. Indoprofen was found to increase the production of luciferase from an SMN2 minigene construct in both C33a and NSC34 cells, without increasing to the same extent the production of luciferase from an SMN1 minigene construct in C33a and NSC34 cells. The splicing construct is as described above; the construct contains exons 6, 7, and an N-terminal fragment of exon 8 fused to a luciferase gene. The pre-mRNA transcript expressed from the construct can be translated to produce luciferase if exon 7 is spliced into the mRNA transcript. If exon 7 is spliced out of the transcript, the reading frame is shifted, and no luciferase protein is produced. One nucleotide can be inserted into exon 7 to eliminate the natural stop codon. The invention provides a high-throughput cell-based screening assay to identify additional compounds that, like indoprofen, can enhance full-length SMN2 protein production. This assay can utilize SMN1-luciferase and SMN2-luciferase splicing cassettes to monitor desired splicing events. Other reporter genes can be used, such as genes encoding the fluorescent proteins, GFP and YFP.

For example, when luciferase is used as the reporter gene in an SMN splicing assay, synthesis of full-length protein can be monitored by luminescence; positive drug candidates will elicit an increase in the amount of luminescence produced from an SMN2-luciferase cassette, which is normally inefficiently spliced, but will not affect the level of luminescence in cells expressing the SMN1-luciferase reporter (because SMN1 transcripts are normally spliced to include exon 7 about 100% of the time). ELISA, Western blotting or gem counts (obtained using indirect immunofluorescence microscopy) can be used to verify the increase in SMN2-luciferase protein levels, and analysis by RNAase protection, Northern blot or RT-PCR, such as quantitative or semiquanititative, can be used to determine whether the effect of the compound is at the level of splicing or mRNA stability. Identification of a role for indoprofen or analogs of this compound in SMN splicing in vitro can be achieved through the generation of stable cell lines expressing the SMN-reporter gene (e.g., SMN-luciferase) splicing cassette. For example, the embryonic mouse spinal cord cell line NSC34 and the human cervical carcinoma cell line C33A can be used for the methods of the invention. NSC34 cells are produced by fusion of motor neuron enriched, embryonic mouse spinal cord cells, with mouse neuroblastoma and are known to express motor-neuron specific proteins (Cashman et al. Dev. Dyn. 194:209-21, 1992). Indoprofen, for example, was shown to enhance SMN2 full-length protein production in both the C33A cell line and the NSC34 cell line (see infra).

An effect of indoprofen or other drugs on SMN splicing in whole animal systems can be achieved through the generation of non-human transgenic animals, e.g., mice, with an SMN1-YFP splicing cassette and an SMN2-GFP splicing cassette. Other optional fluorescent reporter genes include but are not limited to red fluorescent protein (RFP), cyan fluorescent protein (CFP), and blue fluorescent protein (BFP), or any paired combination thereof, provided the paired proteins fluoresce at distinguishable wavelengths. Changes in splicing pattern can be monitored in situ by alterations of the ratio between fluorescence, such as YFP and GFP fluorescence. To determine dosage requirements, the transgenic animals can be administered (e.g., fed or injected with) drugs at different concentrations. The fluorescence intensity ratio (e.g., of GFP/YFP) can be quantified from different tissues, such as neural tissues (e.g., spinal cord and/or brain tissue), using quantitative fluorescence microscopy, to determine the minimal amount of drug required to generate the optimal enhancement of full-length SMN2 production. The splicing pattern of SMN2 can be verified by RNAse protection, Northern blot, or RT-PCR, and protein levels can be monitored by, e.g., Western blotting or immunofluorescence. A test of mRNA stability can also be performed using Northern analysis, a pulse/chase assay, or variety of other molecular biology techniques.

In vivo tests of indoprofen or its analogs can be performed in smn^{-/-} mice transformed with the human hSMN2 gene (unlike humans, mice have only one copy of the SMN gene). These SMA mice, homozygous for a low copy number insertion of the SMN2 transgene, normally die within a few days after birth. Thus, they can provide a model system to determine the therapeutic potential of the identified compounds. SMA mice that are heterozygous for a low copy number insertion of the SMN2 transgene die in utero. The SMA mice or their mothers can be administered indoprofen or another drug (e.g., a candidate drug) by feeding or injection, such as into the spinal cord, during embryonic development or postnatally. Any clinical phenotype or pathological manifestation can then be examined as an indicator of how effectively it can modify the splicing pattern, and appropriate dosage can be determined. The splicing pattern of hSMN2 in different tissues can be determined by RNAse protection, Northern blotting, or, RT-PCR, or mRNA stability can be examined, such as by Northern blot or pulse/chase analysis. SMN protein expression can be determined, for example, by Western blot or immunofluorescence.

The invention provides a method to treat individuals carrying a genetic abnormality that results in a disease characterized by an altered splicing pattern. For example, the invention provides a method to treat the neuromuscular disease SMA. SMA results from a mutation in or a complete deletion of the SMN1 gene, the result of which is a loss of full-length SMN protein normally produced from this gene. By administering indoprofen or another drug, the amount of SMN protein produced in the cell is increased due to the modified splicing pattern of the SMN2 RNA. These drugs can be administered according to the methods of the present invention to an individual at risk for or having SMA. These and other drugs can also be used to treat a variety of other diseases characterized by an altered splicing pattern (see Table 1, for example).

The invention provides a high-throughput assay to identify a drug (e.g., a compound or molecule) capable of increasing SMN2 full-length protein production. This effect can occur in at least three different ways: 1) stimulation of the SMN2 promoter, 2) stimulation of the incorporation of exon 7 into the SMN transcripts, or 3) stabilization of the SMN2 transcripts. A variety of molecular biology assays including RT-PCR and Northern blot analysis can distinguish between the effects of a compound on SMN2 expression and RNA processing.

The high-throughput assay requires the use of cell culture lines, such as mammalian cell lines (e.g., human cell lines). For example, the human cervical carcinoma cell line C33A (ATCC# HTB-31), or the neuronal cell line NSC34 can be used for screening. SMA is a motor neuron disease, and thus analysis of a drug's ability to modify splicing of a splicing reporter construct in motor neurons can facilitate analysis of the drug's effectiveness. The NSC34 cell line was produced by the fusion of motor neuron-enriched embryonic mouse spinal cord cells with mouse neuroblastoma and NSC34 cells were shown to express motor neuron specific proteins (Cashman et al. Dev. Dyn 194:209-21). These qualities make this cell line ideal for testing the efficacy of compounds on SMN2 expression and processing. NSC34 cells are slow growing, and generally more difficult to work with than are C33A cells, and therefore, C33A cells may be a preferred cell line for use in screening assays, particulary for use with the SMN splicing constructs described herein.

For screening, C33A (or NSC34) cells can be used to create two stably transfected cell lines. One cell line can be transformed with an SMN1 fragment/reporter gene construct, such as an SMN1 fragment/luciferase reporter gene construct, and the other cell line can be transformed with an SMN1 fragment/reporter gene construct, such as an SMN2 fragment/luciferase reporter gene construct. The SMN portion of these constructs is described in Lorson et al. (Proc Natl. Acad. Sci. USA 96:6307-6311, 1996), hereby incorporated by reference. The luciferase portion of the construct can be obtained from commercial sources. In an exemplary application, the SMN fragment incorporates exons 6-8 (including all or part of the intervening introns) of SMN1 or SMN2 (Zhang et al., Gene Therapy 8:1532-1538, 2001). These cells (˜35-40M cells/flask) are cultured in culture media supplemented with 10% Fetal Bovine Serum, 50 units/mL penicillin, 50 μg/mL streptomycin sulfate and 400 mg/L G418 (i.e. Geneticin, Gibco, cat. No. 11811-031). Cells are trypsinized and removed from the culture flasks, and quenched with assay medium. The cells are then spun down and re-suspended in assay medium to a concentration of about 100,000; 200,000; 300,000; or 400,000 cells/mL. Using a Zymark Sciclone ALH or similar robot, cells can then be seeded into 384-well assay plates, for a cell density of about 5000; 8000; 11,000; 15,000; or 20,000 cells/well. In the initial round of screening, compounds can be added to the cells to a final concentration of 5-20 μM, preferably, 10 μM, again using the Zymark Sciclone ALH or a similar robot. Cells can be incubated with the test compounds at 37° C. for about 34, 36, 48, or 60 hr, and then assay plates can be cooled to room temperature for ˜10-60 minutes. The medium and compounds can then be aspirated from the wells, followed by a series of washes (e.g., 6 rinse/aspirate cycles) with a PBS solution.

The assay medium used to perform the luciferase assay can be identical to the growth medium described herein, except the G418 selection agent can be omitted. To perform the luciferase assay, a single reagent (25 mM glycylglycine, 15 mM magnesium sulfate, 4 mM EGTA, adjusted to pH 7.85, to which is added 10 μM D-luciferin (Sigma L9504), 10 mM dithiothreitol (DTT), 2 mM adenosine 5′-triphosphate (ATP), 50 μM Coenzyme A, sodium salt and 1.5% Triton X-100) can be used to lyse the cells and to initiate the luciferase reaction. The pH of the lysing/detection reagent is critical for maximizing luminescence. The pH can range from 7.5 to 8.2. For example, the pH can be 7.5, 7.8, 8.0 or 8.2. Preferably the pH of the reagent can be 7.85-7.90. The final substrate concentration (i.e., D-luciferin, can range from 0.01-20 μM. For example, the substrate concentration can be 0.01, 0.1, 1.0, 5, 10, 15 or 20 μM. The final concentration of DTT can be about 10 mM. The concentration of coenzyme A can be 20, 40, 60, or 100 μM, preferably 50 μM. Depending on the reporter gene used in the assay of the invention, the specific reagent composition can vary.

To perform the assay, 53 μL of the luciferase assay reagent is added to each of wells and the plate is immediately transferred to a luminescence plate reader such as the Packard Fusion instrument. In one embodiment, the assay plate is white. The plate can be shaken for 10-240 min, preferably, 30 minutes prior to reading.

To analyze the luminescence data, the dark count background should first be subtracted from all luminescence readings. The signal from each compound-treated well can be normalized by dividing the average of approximately 48 control wells on the same 384-well plate. These control wells can contain cells that have not been treated with any compound. Each compound library plate should be tested multiple times (in triplicate, for example) with each of the two cell lines and the entire assay being repeated in two separate screening events. These exemplary conditions will yield six readings for each compound for each cell line. The highest and lowest of the data readings (i.e., the six normalized readings) can be discarded and the remaining four readings averaged. The average values of compound-treated luminescence versus control luminescence represent baseline luminescence levels. The difference of the luminescence signals generated from the SMN2 fragment/luciferase and SMN1/luciferase cell lines, [SMN2norm(y)-SMN1norm(y)], is the measure of the selective change in SMN2 luciferase expression versus change in SMN1 luciferase production upon treatment with compound y.

After a drug or group of drugs has been identified as causing increased luminescence, they can be retested in a dilution series (such as a 2 fold, 20-point dilution series) with a maximum concentration of 10-100 μM, preferably, 10 μM. These dilutions can be created using a Zymark Sciclone ALH Z-8 or similar robot. The assays can be performed as described for the screening process with the exception of the creation of the dilution series.

Additional assays can be performed to determine the effect of the compound on gene expression, RNA processing, and RNA stability. Exemplary assays can include RT-PCR, for example, which can reveal an effect on SMN2 pre-mRNA splicing of gene expression levels, and Northern and Western analysis, which can reveal an effect on gene expression and mRNA and protein stability.

To further assess the in vivo effect of a compound identified in the screen described herein, the compound can be administered to a transgenic animal, such as mouse, which carries the SMN1 and/or SMN2 splicing cassettes fused to reporter genes such as fluorescent reporter gene. SMN1-YFP and SMN2-GFP are exemplary splicing cassettes. As with the in vitro luciferase assays, the splicing cassettes can incorporate exons 6-8 (and part or all of the intervening introns) of SMN1 and SMN2. Transgenic mice can be administered, such as by feeding or injection, the candidate drugs identified in the high-throughput assay described herein. Effects on full-length protein production can be monitored in different tissues by assaying for alterations in the ratio of fluorescent intensities, e.g., between YFP and GFP signal. Signal can be determined in situ by quantitative fluorescence microscopy. By these methods, the effects of the drugs in different tissues can be assayed separately. These mice can also be used to test the effect of different dosage amounts on protein expression. The ratios of fluorescent intensities, e.g., GFP/YFP intensities, are quantified from neural tissue including spinal cord, brain and other tissues using quantitative microscopy to determine the minimal amount required to generate the optimal enhancement of exon 7 inclusion. RNA isolated from different tissues during different developmental stages can be analyzed by RNAse protection, Northern blot, or, preferably, RT-PCR to determine if, when, and where the compounds effect SMN2 pre-mRNA splicing. SMN2 expression and mRNA stability can be also monitored by methods including Northern analysis.

In vivo tests of indoprofen or its analogs can be performed in smn^{-/-} mice transformed with the human hSMN2 gene (unlike humans, mice only have one copy of the SMN gene). These SMA mice normally die within a few days after birth or during development in utero. After treating them with indoprofen or another drug (for example, by feeding, oral gavage or spinal cord injection) any clinical phenotype or pathological manifestation can be examined as an indicator of the ability to modulate splicing of the gene of interest, or as an indicator of appropriate dosage. The splicing patterns of hSMN2 in different tissues can be determined, e.g., by RNAse protection, Northern blot, or, preferably, RT-PCR. SMN protein expression is determined, e.g., by Western blot and immunofluorescence. SMN2 expression and mRNA stability can also be monitored by methods including Northern analysis.

The methods described above for identifying and testing a drug to treat SMA, can be applied to other diseases characterized by aberrantly spliced genes (see Table 1). The design of appropriate splicing cassettes and use of appropriate cell culture lines will be unique to each disease-related gene. However, the assay method of the invention is generally applicable for monitoring the effect of a test drug on splicing substrates using a reporter gene, such as one that encodes a luminescent or fluorescent protein. The assay method described herein is particularly amenable for high-throughput applications.

Effective Dose

Toxicity and therapeutic efficacy of a drug disclosed herein can be determined by standard pharmaceutical procedures, using either cells in culture or experimental animals to determine the LD50 (the dose lethal to 50% of the population) and the ED50 (the dose therapeutically effective in 50% of the population). The dose ratio between toxic and therapeutic effects is the therapeutic index, which can be expressed as the ratio LD50/ED50. Drugs that exhibit large therapeutic indices are preferred. While drugs that exhibit toxic side effects may be used, care should be taken to design a delivery system that targets such drugs to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (that is, the concentration of the test compound which achieves a half-maximal inhibition of symptoms) as determined in cell culture. Such information can be used to more accurately determine useful doses in humans. Levels in plasma may be measured, for example, by high performance liquid chromatography or by liquid chromatography-mass spectrometry (LC-MS)

Formulations and Use

Pharmaceutical compositions for use in accordance with the present invention can be formulated in a conventional manner using one or more physiologically acceptable carriers or excipients.

Thus, the drugs (e.g., compounds or molecules) and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art. Liquid preparations for oral administration may take the form of, for example, solutions, syrups or suspensions, or they can be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For buccal administration the compositions can take the form of tablets or lozenges formulated in conventional manner.

For administration by inhalation, the drugs for use according to the present invention are conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The drugs can be formulated for parenteral administration by injection, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

The drugs can also be formulated in rectal compositions such as suppositories or retention enemas, for example, containing conventional suppository bases such as cocoa butter or other glycerides.

In addition to the formulations described previously, the drugs can also be formulated as a depot preparation. Such long acting formulations may be administered by implantation (for example subcutaneously or intramuscularly) or by intramuscular injection. Thus, for example, the drugs can be formulated with suitable polymeric or hydrophobic materials (for example as an emulsion in an acceptable oil) or ion exchange resins, or as sparingly soluble derivatives, for example, as a sparingly soluble salt.

The compositions can be presented in a pack or dispenser device that may contain one or more unit dosage forms containing the active ingredient. The pack may, for example, comprise metal or plastic foil, such as a blister pack. The pack or dispenser device can be accompanied by instructions for administration.

The therapeutic compositions of the invention can also contain a carrier or excipient, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. The drugs of the invention can be administered by any standard route of administration. For example, administration can be parenteral, intravenous, subcutaneous, intramuscular, intracranial, intraorbital, opthalmic, intraventricular, intracapsular, intraspinal, intracisternal, intraperitoneal, transmucosal, or oral. A modulatory compound can be formulated in various ways, according to the corresponding route of administration. For example, liquid solutions can be made for ingestion or injection; gels or powders can be made for ingestion, inhalation, or topical application. Methods for making such formulations are well known and can be found in, for example, “Remington's Pharmaceutical Sciences.” It is expected that the preferred route of administration will be intravenous.

It is recognized that the pharmaceutical compositions and methods described herein can be used independently or in combination with one another. That is, subjects can be administered one or more of the pharmaceutical compositions, e.g., pharmaceutical compositions comprising a drug of the invention, in temporally overlapping or non-overlapping regimens. When therapies overlap temporally, the therapies may generally occur in any order and can be simultaneous (e.g., administered simultaneously together in a composite composition or simultaneously but as separate compositions) or interspersed. By way of example, a subject afflicted with a disorder described herein can be simultaneously or sequentially administered both a drug that modifies the splicing pattern of a pre-mRNA and an antibody which can be conjugated or linked with a therapeutic agent, a cytotoxic agent, an imaging agent, or the like. Those skilled in the art will recognize that appropriate dosing and administering regimens can be applied to the other therapeutics listed above.

This invention further pertains to novel agents identified by the above-described screening assays. The test agents can be identified from collections of small molecules, peptides, peptide aptamers and the like. Accordingly, it is within the scope of this invention to further use an agent identified as described herein in an appropriate animal model to determine the efficacy, toxicity, side effects, or mechanism of action, of treatment with such an agent. Furthermore, novel agents identified by the above-described screening assays can be used for treatments as described herein.

Kits

The diagnostic and therapeutic methods to assay the ability of a compound to alter the splicing pattern of a gene can be assembled as kits. Accordingly, for diagnostic purposes, the invention features a one-step luciferase assay kit for detecting the luminescence of a reporter gene. The kit can minimally contain a single reagent that consists of a cell lysing agent and a substrate for reporter enzyme detection. Optionally, instructions for use are included. The reagent, for example, can contain a D-luciferin substrate for the detection of a luciferase reporter enzyme. The substrate concentration can range from 0.01-20 μM. For example, the luciferin substrate concentration can be 0.01, 0.1, 1.0, 5, 10, 15 or 20 μM. Preferably, the substrate concentration is less than 20 μM. The pH of the reagent can range from 7.5 to 8.2. For example, the pH can be 7.5, 7.8, 8.0 or 8.2, preferably 7.9. The luciferin reagent mix preferably contains coenzyme A and dithiothreitol (DTT). The concentration of DTT can range from 4 mM to 15 mM. Preferably, the DTT concentration is 10 mM. The concentration of coenzyme A can range from 20-100 μM; preferably the coenzyme A concentration is 50 μM. The reagent of the kit can be stored lyophilized or in solution. Optionally, the kit can include cells, e.g., mammalian cells, e.g., mouse, human or primate cells, for use in the assay. The kit can also include a vector encoding a luminescent protein flanked at its 5′ end by a multiple cloning site. The vector should be suitable for expression in bacteria and cell culture, e.g., mammalian cell culture. The kit can also include assay plates. The plates can be white, for example. A white assay plate provides optimal assay sensitivity because it reflects light towards the detection instrument.

A kit of the invention can also contain a control sample or a series of control samples that can be assayed and compared to the test sample. Each component of the kit can be enclosed within an individual container, and all of the various containers can be within a single package.

The kits disclosed herein can be used to determine whether a drug can affect the splicing pattern of a test transcript fused to a reporter gene encoding a luminescent protein, e.g., luciferase. In this case, the test transcript would contain at least two exons separated by an intron. The test transcript can alternatively consist of three exons, with two intervening introns, the third exon fused to the reporter gene.

The assay described herein can be used to measure gene expression, e.g., transcription and translation, or the activity of a genetic regulatory element. In these cases, a nucleic acid, e.g., a gene, gene fragment or regulatory element can be fused to the 5′ end of the reporter gene. Up or down regulation of gene expression and/or of a regulatory element can be monitored as indicated by reporter protein activity, such as luminescence or fluorescence, and the like. The one-step method described herein to detect luminescence can be used in this assay.

This invention is further illustrated by the following example, which should not be construed as limiting. The teachings of all references, patents and published patent applications cited throughout this application are incorporated herein by reference.

### EXEMPLIFICATION

### Assay for Identification of Compounds that Stimulate Inclusion of Exon 7 in SMN2 mRNA

In a screen to identify compounds capable of enhancing the inclusion of exon 7 in SMN2 mRNA, cells of the human cervical carcinoma cell line C33A (ATCC#HTB-31) were stably transformed with an SMN fragment/luciferase reporter gene construct that incorporated exons 6-8 of SMN1 or SMN2 for use in a high-throughput screening assay. These cells were cultured in Dulbecco's Modified Eagle's Medium (DMEM) with 10% Fetal Bovine Serum (Sigma F 2442), 50 units/mL penicillin 50 μg/mL streptomycin sulfate and 400 mg/L G418 (i.e. Geneticin, Gibco, cat. No. 11811-031). The assay medium used to perform the luciferase assay was identical to the growth medium except the G418 selection agent was omitted. To perform the luciferase assay, a single reagent (25 mM glycylglycine, 15 mM magnesium sulfate, 4 mM EGTA, adjusted to pH 7.85, to which is added 10 μM D-luciferin (Sigma L9504), 10 mM dithiothreitol (DTT, Roche Molecular Biochemicals 100034), 2 mM adenosine 5′-triphosphate (ATP, Sigma A7699), 50 μM Coenzyme A, sodium salt (Sigma C 3144) and 1.5% Triton X-100) was used to lyse the cells and to initiate the luciferase reaction.

Using this cell-based luminescence assay, a screen was performed to identify compounds that modulate SMN splicing. Stably transfected C33A cells (˜35-40 M cells/flask) were cultured, then removed from T-175 culture flasks with 3 mL trypsin, and quenched with 7 mL assay medium. The cells were spun down and re-suspended in assay medium to a concentration of 200,000 cells/mL. Cells were immediately seeded in 384-well assay plates (Nalge Nunc white plates, #164610) 57 μL/well for a cell density of 11,000 cells/well. Compounds from a library obtained from the National Institute of Neurological Disorders and Stroke (NINDS) were added to the cells to a final concentration of 10 μM (diluted with assay medium by 1000× from the 10 mM stock solution in DMSO). The cells were incubated with the test compounds at 37° C. for 48 hr. A Zymark Sciclone ALH was used for cell seeding and compound addition. After incubation, the assay plates were cooled to room temperature for 10-60 minutes. The medium and compounds were aspirated from the wells, followed by 6 rinse/aspirate cycles with a PBS solution. 53 μL of the luciferase assay reagent was added and the plate was immediately transferred to a luminescence plate reader. The plate was shaken for 30 minutes prior to reading. The plate processing steps were performed on an integrated Packard Minitrak/Sidetrak platereader.

To analyze the data, the dark count background was first subtracted from all luminescence readings. The signal from each compound treated well was divided by the average of 48 control wells on the same 384-well plate. Each compound library plate was tested in triplicate with each of the two cell lines, and the entire assay was repeated on two separate occasions, yielding six readings for each compound for each cell line. The highest and lowest of the six normalized readings were dropped and the remaining four readings averaged. These average values of compound-treated luminescence versus control luminescence are denoted “Xnorm(y)”, where X indicates the cell line, SMN1-luciferase or SMN2-luciferase, and y represents the compound. The difference of [SMN2norm(y)-SMN1norm(y)] measures the selective change in SMN2-luciferase expression versus change in SMN1 luciferase production upon treatment with compound y. This is the final score that is output.

By this method, 1400 compounds/drugs from a chemical library were screened and 48 compounds were identified as having increased luminescence, and thus were selected for retesting in a 2-fold, 20-point dilution series with a maximum concentration of 10 uM. These dilutions were created with a Zymark Sciclone ALH Z-8. Once created, the assay was performed as described for the screening process. Concentrations required to achieve 50% of the maximally achievable effect for each compound (“EC50”) were calculated using Xlfit (IDBS).

The 48 potential hits were also retested by RT-PCR. In this secondary assay, primers were used to amplify both the correctly spliced and the incorrectly spliced mRNA transcripts of the endogenous SMN2 gene. Compounds that improved the ratio of these two transcripts (increasing the proportion of transcripts that include exon 7) were identified in this secondary screen.

Results

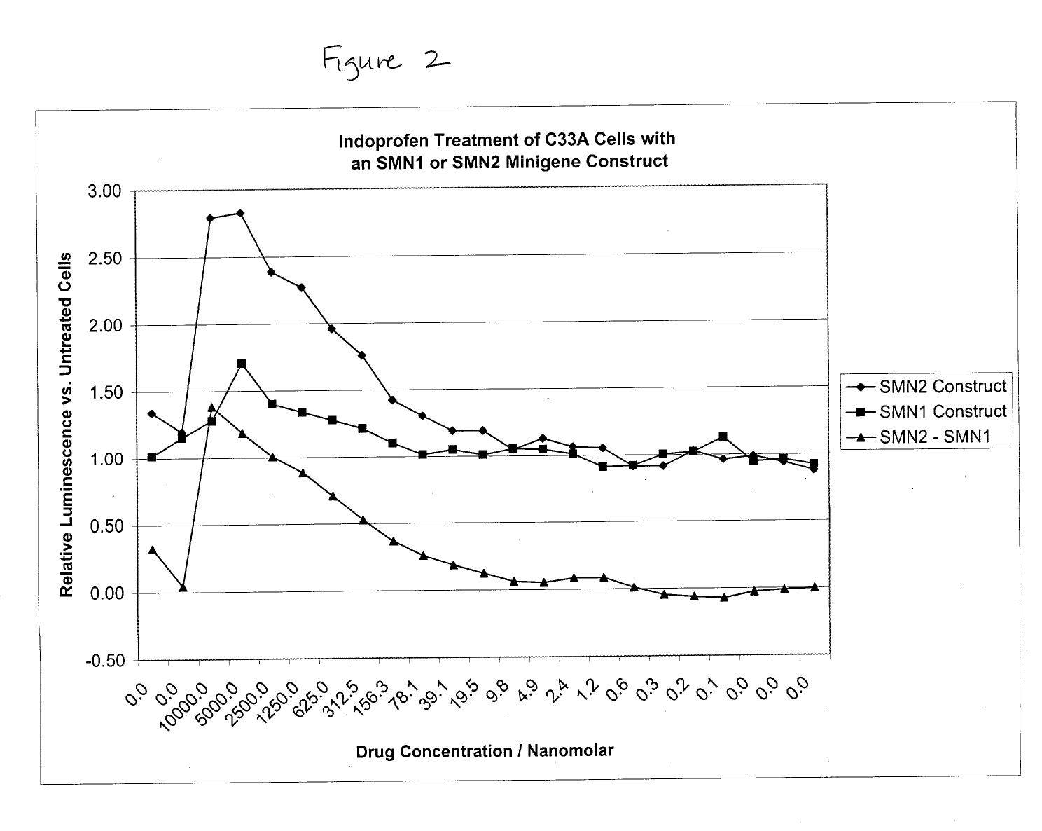

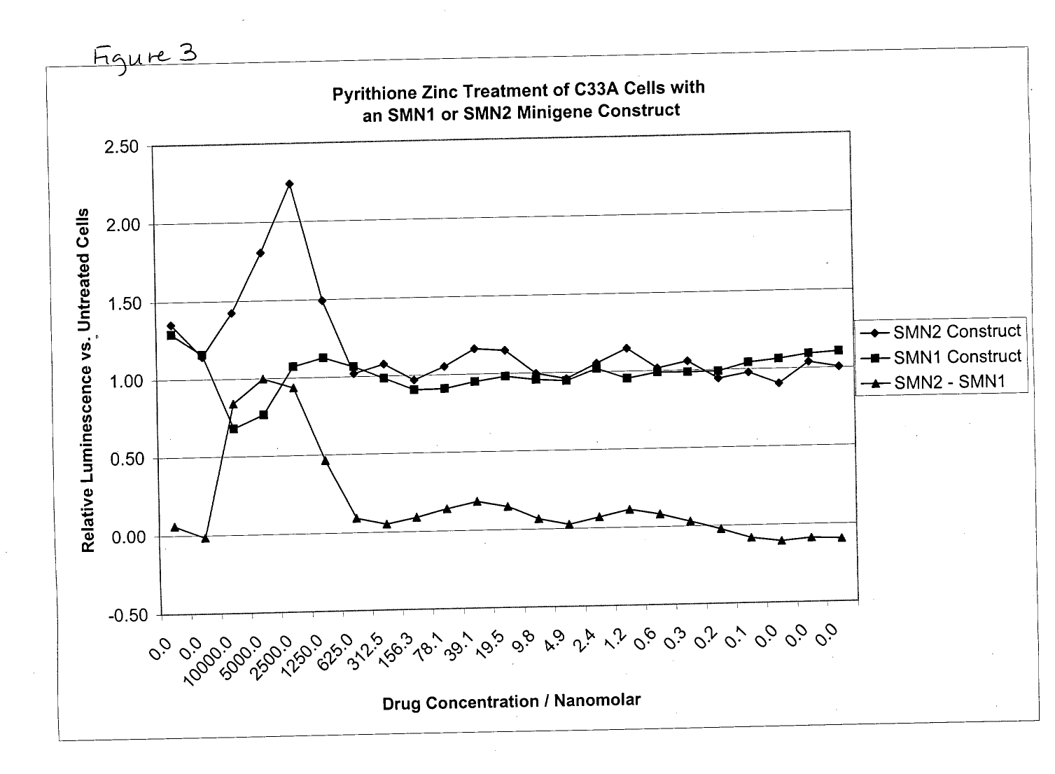

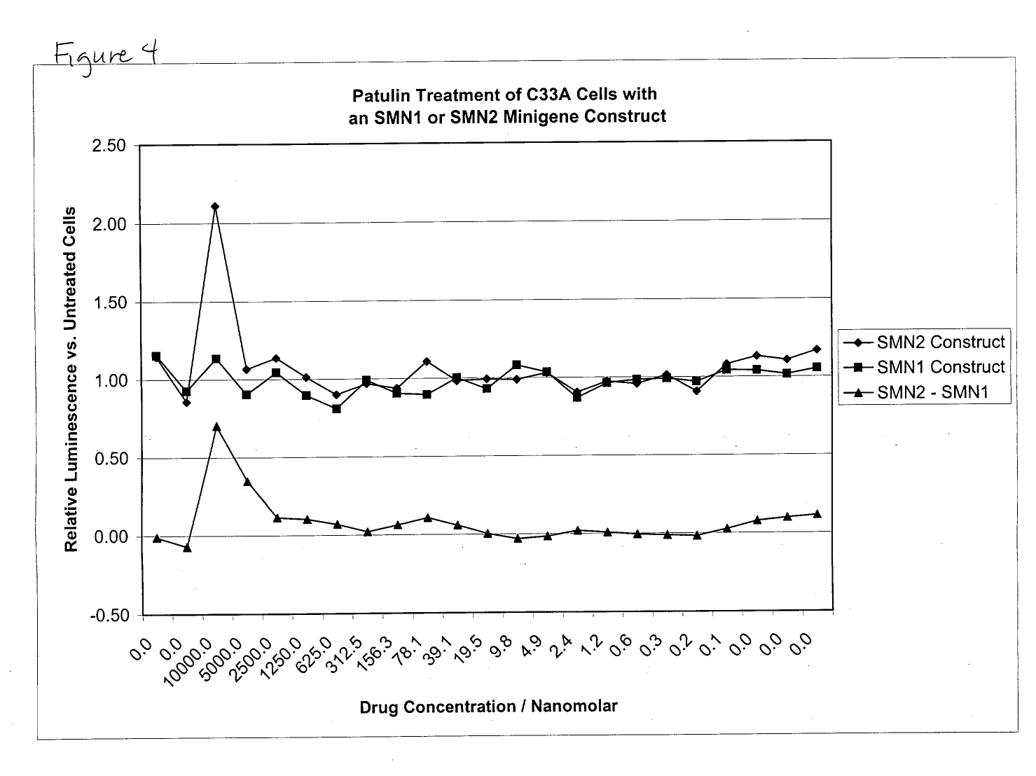

Compounds that exhibited increased luminescence in C33A cells were indoprofen (Sigma-Aldrich); pyrithione zinc (Sigma-Aldrich); patulin (Sigma-Aldrich); camptothecin (Sigma-Aldrich); cefoxitin sodium (Sigma-Aldrich); and amantadine (Sigma-Aldrich). Indoprofen was the only compound that also caused an increased luminescence in NSC34 cells transformed with the SMN constructs of the invention. FIGS. 2-4 are graphs that illustrate the effect of three of these drugs on relative luminescence produced from SMN2-luciferase (diamonds) and SMN1-luciferase (squares) constructs expressed in C33A cells. The lines marked with triangles represents values for treated cell luminescence/control cell luminescence. This value is representative of the increase in luminescence in the SMN2-luciferase transformed cells minus the increase in luminescence for the SMN1-luciferase transformed cells. The “highest point” on the line marked with triangles is a reflection of the selective change in SMN2-luciferase expression versus change in SMN1 luciferase production upon treatement with the drug. These values are shown in Table 2. A higher value in Table 2 indicates that the drug affected the SMN2 expression to a greater extent than SMN1 expression. Thus the drug is a candidate for one that can promote inclusion of exon 7 in the SMN transcript of the SMN2 (and SMN1) reporter construct. Because the SMN1 reporter construct is already including exon 7 approximately 100% of the time, no increase in luminescence is observed.

TABLE 2
Drugs that cause a selective change in SMN2 expression versus change
in SMN1 luciferase production

| Drug | SMN2 luminescence/SMN1 luminescence |
|---|---|
| Indoprofen | 1.4 |
| Pyrithione zinc | 1.0 |
| Patulin | 0.7 |
| Camptothecin | 0.4 |
| Cefoxitin sodium | 0.3 |
| amantadine | 0.3 |

A number of non-steroidal anti-inflammatory drugs were also screened for an effect on SMN splicing in the assay. These compounds included ibuprofen, ketoprofen flurbiprofen, fenoprofen, suprofen, aspirin and acetominophen. None of these compounds resulted in increased luminescence from the SMN2-luciferase reporter construct.

Table 3 shows indoprofen analogs that were tested in the assay of the invention, but which did not have a specific effect on splicing of the SMN2-luciferase cassette.

TABLE 3
Analogs of indoprofen did not specifically alter splicing of the SMN2-luciferase
reporter construct

| | | | | Effect on splicing |
|---|---|---|---|---|
| | | | Effect on splicing of | of SMN1- |
| | | | SMN2-luciferase | luciferase control |
| Name | Source | CAS# | in C33A cells | in C33A cells |
| 1-tert-butyl-1-hydroxy-2- | Sigma | | None | Not tested |
| methyl-6-phenyl-3- | Cat # S73,343-1 | | | |
| isoindolinone | | | | |
| 3-(1,1-diethylpropyl)-3- | Sigma | | None | Not tested |
| hydroxy-2-methyl-1- | Cat # S76,668-2 | | | |
| isoindolinone | | | | |
| 3-(1,1-diphenylethyl)-3- | Sigma | | None | Not tested |
| hydroxy-2-methyl-1- | Cat. # S73,531-0 | | | |
| isoindolinone | | | | |
| 3-(2,4-hydroxybenzoyl)- | Sigma | | None | Not tested |
| 2-(4-methylphenyl)-1- | Cat. #R66,372-7 | | | |
| isoindolinone | | | | |
| 3-(2,5-hydroxybenzoyl)- | Sigma | | None | Not tested |
| 2-(4-methylphenyl)-1- | Cat. #R66,353-0 | | | |
| isoindolinone | | | | |
| 3-(2-hydroxy-5- | Sigma | | None | Not tested |
| methylbenzoyl)-2-(4- | Cat. #R66,402-2 | | | |
| methylphenyl)-1- | | | | |
| isoindolinone | | | | |
| 3-(4-chlorophenyl)-3- | Sigma | | None | Not tested |
| hydroxy-2- | Cat. #S76,085-4 | | | |
| (tetrahydropyran-2- | | | | |
| yloxy)-1-isoindolinone | | | | |
| 1H-isoindol-1-one,2,3- | Chembridge | 68327-78-6 | Increased | Increased |
| dihydro-2-[4-91- | Corp., San Diego, | | luminescence | Luminescence |
| hydroxyethyl)phenyl] | CA | | (Non-specific effect) | (Non-specific |
| | Order No. | | | effect) |
| | 5235894 | | | |
| 1H-isoindol-1-one,2,3- | Interbioscreen | 480-91-1 | None | None |
| dihydro- | Ltd., Moscow, | | | |
| | Russia | | | |
| | STOCK1N-30308 | | | |
| 1-(4-Chlorobenzoyl)-5- | BioMol, Plymouth | 53-86-1 | None | None |
| methoxy-2-methyl-3- | Meeting, PA; | | | |
| indoleacetic acid | #EI-131 | | | |
| 2-Phthalimidino-glutaric | AG Scientific, San | 26577-32-2 | None | None |
| acid | Diego, CA; | | | |
| | #P-1187 | | | |
| 1H-isoindol-1-one,2,3- | Interbioscreen | 5388-42-1 | None | None |
| dihydro-2-phenyl | Ltd., Moscow, | | (also no effect in | (not tested in |
| | Russia | | NSC34 cells) | NSC34 cells) |
| | STOCK1N-30308 | | | |
| Alpha- | Aldrich Chemical | 1009-67-2 | Decreased | Decreased |
| methylhydrocinnamic | Co., Milwaukee, | | luminescence | luminescence |
| acid | WI; | | (no effect in NSC34 | (no effect in |
| | #39,0152-2 | | cells) | NSC34 cells) |
| 2H-isoindol-2-butanoic, | Chembridge | 3130-75-4 | None | None |
| 1,3-dihydro-1,3-dioxo | Corp., San Diego, | | (also no effect in | (also no effect in |
| | CA; | | NSC34 cells) | NSC34 cells) |
| | #5107302 | | | |
| 2H-isoindol-2-acetic acid, | Ambinter, Paris, | 96017-10-6 | None | None |
| 1,3-dihydro-1-oxo-alpha- | France; | | (also no effect in | (also no effect in |
| (phenylmethyl)- . . . | #8L-018 | | NSC34 cells) | NSC34 cells) |
| 2H-isoindol-2-propanoic | Ambinter, Paris, | 439095-70-2 | None | None |
| acid, 1,3-dihydro-beta-(4- | France; | | (also no effect in | (also no effect in |
| methylphenyl)-1-oxo | #9R-0613 | | NSC34 cells) | NSC34 cells) |
| Methyl 2-[4-(1,3-dioxo- | Ryan Scientific, | | None | Not tested |
| 2,3-dihydro-1H-isoindol- | Isle of Palms, Sc; | | | |
| 2-yl)phenyl . . . | Cat. #JFD 02149 | | | |
| Methyl 2-[4-(1,3-dioxo- | Ryan Scientific, | | None | Not tested |
| 2,3-dihydro-1H-isoindol- | Isle of Palms, SC; | | | |
| 2-yl)-3-me . . . | Cat. #JFD 02151 | | | |
| Chemical formula: | Ryan Scientific, | | None | Not tested |
| C16H13NO2 | Isle of Palms, SC; | | | |
| | Cat. #1S-09269 | | | |
| Chemical formula: | Ryan Scientific, | | None | Not tested |
| C17H15NO2 | Isle of Palms, SC; | | | |
| | Cat. | | | |
| | #BAS0161180 | | | |

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating an individual having or at risk for having Spinal Muscular Atrophy (SMA) by administering a compound having the formula: 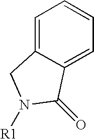
where R1 is selected from the group consisting of:
C₁-C₂₅ alkyl, arylalkyl, substituted alkyl or substituted arylalkyl;
C₃-C₂₅ cycloalkyl, substituted cycloalkyl; arylcycloalkyl, or substituted arylcycloalkyl;
C₁-C₂₅ alkenyl, arylalkenyl, substituted alkenyl or substituted arylalkenyl; C₃-C₂₅ cycloalkenyl, arylcycloakenyl, substituted cycloalkenyl, or substituted arylcycloalkenyl;
C₁-C₂₅ alkynyl, arylalkynyl, substituted alkynyl, or substituted arylalkynyl;
—COOH;
—COOR7, where R7 is independently selected from the same moieties as R1; and
—CONH₂.

2. The method of claim 1 in which R1 is a residue having the following formula: 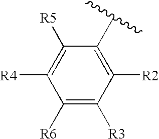
where each of R2, R3, R4, R5, and R6 is independently selected from hydrogen;
C₁-C₂₅ alkyl, arylalkyl, substituted alkyl or substituted arylalkyl;
C₃-C₂₅ cycloalkyl, substituted cycloalkyl; arylcycloalkyl, or substituted arylcycloalkyl;
C₁-C₂₅ alkenyl, arylalkenyl, substituted alkenyl or substituted arylalkenyl; C₃-C₂₅ cycloalkenyl, arylcycloakenyl, substituted cycloalkenyl, or substituted arylcycloalkenyl;
C₁-C₂₅ alkynyl, arylalkynyl, substituted alkynyl, or substituted arylalkynyl;
—COOH;
—COOR7, where R7 is independently selected from the same moieties as R1; and
—CONH₂.

3. The method of claim 2 in which R6 is a C1-C5 alcohol, a C1-C5 carboxylic acid, a C1-C5 ester, a C1-C5 aldehyde, or a C1-C5 ketone.

4. The method of claim 3 in which R6 is C3 carboxylic acid.

5. The method of claim 4 in which R6 is

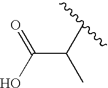

6. The method of claim 3 in which R6 is a C1-C6 alcohol.

7. The method of claim 6 in which R6 is a C3 alcohol.

8. The method of claim 7 in which R6 is

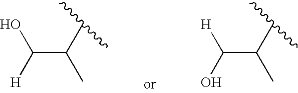

9. The method of claim 1, wherein the compound is indoprofen.

10. The method of claim 1, wherein the individual has a deletion of or a mutation in the SMN1 gene.

11. The method of claim 1, wherein the administration results in an increase in functional SMN2 protein levels.

12. A method for treating an individual having or at risk for having SMA by administering a compound selected from the group consisting of indoprofen; pyrithione zinc; patulin; camptothecin; cefoxitin sodium; and amantadine.

13. The method of claim 12, wherein the individual is treated with indoprofen.

14. The method of claim 12, wherein the individual has a deletion of or a mutation in the SMN1 gene.

15. The method of claim 12, wherein the administration results in an increase in functional SMN2 protein levels.

16. The method of claim 15, wherein the administration results in an increase in levels of SMN2 that include exon 7.

17. The method of claim 15, wherein the administration results in an increase in SMN2 protein levels in the cytoplasm and the nucleus.

18. The method of claim 15, wherein the administration results in an increase in nuclear SMN2 protein levels localized to gems and Cajal bodies.

19. The method of claim 15, wherein the administration results in an increase in levels of SMN2 oligomers.

20. A method of identifying a drug as a candidate for treating a disease, wherein the disease is characterized by an altered splicing pattern of an RNA, said RNA comprising a first exon, a second exon and an intron between the first and second exon, said method comprising:
a) providing a cell comprising a transcribable cassette comprising DNA encoding the first and second exon and the intron, DNA encoding said second exon being fused to DNA encoding a reporter protein;
b) exposing the cell to a candidate drug;
c) lysing the cell by exposing it to a mixture comprising a lysing agent and a substrate for the reporter protein; and
d) detecting the reporter protein as an indication that the protein product comprises the second exon, wherein detection of the reporter protein is an indication that the RNA is spliced to produce an intact open reading frame,
thereby identifying a drug as a candidate for treating a disease characterized by an altered RNA spliing pattern.

21. The method of claim 20, wherein the transcribable cassette further comprises DNA encoding a third exon and an intron between the second and third exon, and wherein
the DNA encoding the reporter protein is fused to the 3′ end of the third exon instead of the second exon.

22. The method of claim 20, wherein the reporter protein is a fluorescent or luminescent protein.

23. The method of claim 20, wherein the reporter protein is luciferase, GFP or YFP.

24. The method of claim 20, wherein the RNA is an SMN2 RNA.

25. The method of claim 20, wherein the RNA is from a gene selected from the group consisting of NF1; ATM; fibrillin (FBN1); dystrophin (DMD); BRCA1; TP53; MAPT; CD44; and CDKN2A.

26. The method of claim 20, wherein the RNA and the cassette further comprise a third internal exon, located between the first and second exons.

27. The method of claim 20, wherein the substrate is D-luciferin and the reporter protein is luciferase.

28. The method of claim 27, wherein the substrate is present at a concentration less than 20 μM.

29. The method of claim 27, wherein the pH of the mixture comprising the lysing agent and the substrate is between about 7.5 and about 8.2.

30. The method of claim 29, wherein the pH of the mixture comprising the lysing agent and the substrate is about 7.9.

31. The method of claim 27, wherein the lysing agent comprises coenzyme A.

32. The method of claim 27, wherein the lysing agent contains dithiothreitol (DTT) at a concentration of from about 8 mM to about 16 mM.

33. The method of claim 32, wherein the concentration of DTT is about 10 mM.

34. The method of claim 27, wherein the number of cells is from about 10,000 to about 20,000 per well of a 384-well plate.

35. The method of claim 27, wherein the number of cells is from about 40,000 to about 80,000 per well of a 96-well plate.

36. The method of claim 27, wherein incident light is excluded from the cell for a period of three hours prior to initiating the method.

37. The method of claim 20, wherein the cell is from an embryonic mouse spinal cord cell line, NSC34, or a human cervical carcinoma cell line, C33A.

38. The method of claim 20, wherein the drug is a non-polymer chemical compound, peptide or aptamer.

39. The method of claim 26, wherein the first exon is exon 6, the second exon is exon 8 and the third exon is exon 7 of an SMN1 or SMN2 gene.

40. The method of claim 20, further comprising
d) administering the drug to a nonhuman transgenic animal in whose cells an aberrantly spliced transcript of the RNA is expressed; and
e) detecting a modification in the amount of a spliced isoform of the transcript,
wherein detection of a modification in the amount of the spliced isoform indicates whether the drug is a positive candidate to treat the disease.

41. The method of claim 40, wherein the non-human transgenic animal is a mouse, rat, primate, sheep, dog, cow, goat or chicken.

42. A pharmaceutical composition comprising a drug identified by the method of claim 20, or a pharmaceutically acceptable salt or derivative thereof in association with a pharmaceutically acceptable diluent or carrier.

43. An in vivo method of identifying a drug for treating a disease, wherein the disease is characterized by an altered splicing pattern of a human gene, said gene comprising a first exon, a second exon and an intron between the first and second exons, said method comprising,
a) providing a transgenic animal comprising a transcribable cassette comprising the first and second exon and the intron, said second exon being fused to DNA encoding a reporter protein, and administering a candidate drug to the animal; and
b) detecting fluorescence from the fluorescent protein,
wherein a change in fluorescence before and after administration of the drug is an indication that the protein product comprises the second exon,
thereby identifying the drug as a candidate for treating a disease.

44. The method of claim 43, wherein the transgenic animal comprises:
a first transcribable cassette comprising the first and second exon and the intron from a first gene, the second exon being fused to DNA encoding a first fluorescent protein; and
a second transcribable cassette comprising the first and second exon and the intron from a second gene, the second exon being fused to DNA encoding a second fluorescent protein,
wherein detection of a change in the fluorescence ratio between the first and second transcribable cassettes before and after administration of the drug is an indication that the drug has a differential effect on the splicing of the first and second cassettes.

45. The method of claim 44, wherein the first fluorescent protein is YFP and the second fluorescent protein is GFP.

46. The method of claim 43, wherein fluorescence is detected by quantitative fluorescence microscopy.

47. A method of in vivo analysis of a compound as a candidate for treating a disease, wherein the disease is characterized by an altered splicing pattern of a gene, said gene comprising a first exon, a second exon, a third exon located between the first and second exons, a first intron located between the first and third exon, and a second intron located between the third and second exon, said method comprising,
a) providing a transgenic animal that includes a transcribable cassette that includes the first, third and second exons, and first and second introns, said second exon being fused to DNA encoding a reporter protein;
b) exposing the animal to a candidate compound; and
c) detecting the reporter protein,
wherein detection of the reporter protein is an indication that the protein product comprises the third exon and further as an indication of whether the compound is a positive candidate for treating the disease.

48. The method of claim 47, wherein the transgenic animal includes
a first transcribable cassette comprising the first, third and second exons, and the first and second introns from a first gene, the second exon being fused to DNA encoding a first reporter protein; and
a second transcribable cassette comprising the first, third and second exons, and the first and second intron from a second gene, the second exon being fused to DNA encoding a second reporter protein.

49. The method of claim 48, wherein the first and second genes are human SMN1 and SMN2.

50. The method of claim 48, wherein the first or second reporter protein is a first and second fluorescent protein.

51. The method of claim 50, wherein the first and second fluorescent proteins are YFP and GFP.

52. The method of claim 49, wherein the first exon is exon 6 of SMN1 or SMN2, the third exon is exon 7, and the second exon is exon 8.

53. The method of claim 50, wherein fluorescence is detected by quantitative fluorescence microscopy.

54. A method of in vivo analysis of a compound as a candidate for treating a disease, wherein the disease is characterized by an altered splicing pattern of a human gene, said gene comprising a first exon, a second exon and an intron between the first and second exon, said method comprising:
a) providing a transgenic non-human animal that includes a transcribable cassette that includes the first and second exon and the intron;
b) exposing the animal to a candidate compound;
c) detecting a spliced isoform of the gene by RT-PCR; and
d) determining whether the compound is a good candidate for treating a disease based on the effect of the compound on the splicing pattern of the human gene.

55. The method of claim 54, wherein the gene and cassette comprise additional exons.

56. The method of claim 54, wherein the transgenic animal is a transgenic mouse.

57. The method of claim 55, wherein the gene and cassette comprise the complete human SMN2 gene.

58. The method of claim 57, wherein hSMN2 protein production is monitored by Western blot, immunofluorescence, ELISA or cytoblot assays.

59. The method of claim 57, wherein hSMN2 RNA processing is monitored by standard molecular biology techniques, comprising RT-PCR, Northern analysis, and mRNA stability assays.

60. A kit for the in vivo analysis of a compound to modify expression of a target gene, the kit comprising a reagent comprising a lysing agent and a substrate for a luminescent protein.

61. The kit of claim 60, further comprising an instruction manual.

62. The kit of claim 60, wherein the substrate is D-luciferin.

63. The kit of claim 62, wherein the substrate is present at a concentration less than 20 μM.

64. The kit of claim 62, wherein the pH of the reagent is between about 7.5 and 8.0.

65. The kit of claim 64, wherein the pH of the reagent is about 7.9.

66. The kit of claim 62, wherein the reagent contains coenzyme A.

67. The kit of claim 62, wherein the reagent contains DTT at a concentration between 4 mM and 15 mM.

68. The kit of claim 67, wherein the DTT is at a concentration of about 10 mM.

69. A method for performing a business of screening for drugs that modify the splicing pattern of a gene, the method comprising:
(a) accepting orders for performing a screen for a drug that modifies the splicing pattern of a gene designated by a user;
(b) providing an in vitro screening system comprising a transcribable cassette comprising a first and second exon and an intervening intron, wherein the second exon is fused to a reporter gene, and a detection system;
(c) recording the effect of each candidate drug in a library on the expression of the reporter gene in a print or computer-readable medium; and
(d) providing the print or computer-readable medium to the user, thereby performing a business for screening for a drug.

70. The method of claim 69, wherein the library is provided by the user.

71. A method of identifying a drug for treating SMA comprising:
a) providing about 11,000 cells at about 200,000 cells/mL, wherein the cells comprise a transcribable cassette comprising exons 6, 7, and a fragment of exon 8 of the SMN2 gene, wherein exon 8 is fused in-frame to a luciferase gene, and wherein a G is inserted at the 48^{th} position of exon 7, and wherein the cells are not exposed to incident light for three hours prior to exposure to a candidate drug;
b) exposing the cells to a candidate drug;
c) lysing the cells by adding a mixture comprising a lysing agent adjusted to a pH of about 7.85, D-luciferin, 10 mM dithiothreitol, Coenzyme A, and Triton X-100; and
d) detecting luminescence from the luminescent protein,
wherein detection of a modification in luminescence following exposure to the drug is an indication that the drug is a positive candidate for treating SMA.
